(19) United States
(12) Patent Application Publication
(10) Pub. No.: US 2021/0287770 A1
(43) Pub. Date: Sep. 16, 2021

(54) ELECTRONIC PATIENT CREDENTIALS

(71) Applicant: Lumedic Acquisition Co, Inc., Seattle, WA (US)

(72) Inventors: Brian Anderson, Kirkland, WA (US); Christian M. Ingrao, Kirkland, WA (US); Michael W. Nash, Kirkland, WA (US); Benjamin K. Parkison, Redmond, WA (US)

(21) Appl. No.: 17/198,079

(22) Filed: Mar. 10, 2021

Related U.S. Application Data
(60) Provisional application No. 62/987,750, filed on Mar. 10, 2020.

(51) Int. Cl. G16H 10/65; G16H 40/20; G06K 7/14; G06K 19/06; H04L 9/30; G06F 21/45; H04L 29/06; G06F 9/54; H04L 29/08; H04L 12/24

(52) U.S. Cl. CPC G16H 10/65; G16H 40/20; G06K 7/1417; G06K 19/06037; H04L 9/30; G06Q 40/08; H04L 63/083; G06F 9/541; H04L 67/10; H04L 41/046; G06F 21/45

(57) ABSTRACT

An issuer client of a service provider establishes a trusted, private, and cryptographically secured connection with a wallet application of a user through a cloud-based agent and provides credential information to the wallet application via the private connection. The credential information is encrypted using a public key of the wallet application dedicated for the private connection with the cloud-based agent. Upon receiving the encrypted credential information, the wallet application decrypts it using a private key to obtain the credential information. The cloud-based agent digitally signs the credential information using a private key of the issuer client, which can be verified by a public key of the issuer client stored in a public identity ledger.

### CROSS-REFERENCE TO RELATED APPLICATIONS

This application claims the benefit of 62/987,750, filed Mar. 10, 2020 and entitled “ELECTRONIC PATIENT CREDENTIALS,” which is hereby incorporated by reference in its entirety.

In cases where the present application conflicts with a document incorporated by reference, the present application controls.

### BACKGROUND

In the physical world, credentials are part of our day-to-day life and can include everything we can express as a document, e.g., driver license, visa or healthcare coverage programs. For example, the credentials in some cases include information related to identifying the subject of the credential, for example, a photo, name, or identification number; information related to the issuing authority, for example, a city government, national agency, or certification body; information related to the type of credential, for example, a Dutch passport, an American driving license, or a health insurance card; and information related to attributes or properties being asserted by the issuing authority about the subject, for example, nationality, the classes of vehicle entitled to drive, or date of birth; and other credentials.

Today, patients typically navigate the healthcare system with several physical credentials. The first is proof of identification, which often also includes patient demographic information such as address, height and weight, and legal name. In the United States, this is most commonly represented by a state driver's license. For patients with existing health benefits, a second credential is an insurance card. This is an unregulated credential, issued and provided by that patient's insurance benefit provider, which includes information unique to that patient to help healthcare providers access the patient's benefit information.

Depending on the reason for care, the healthcare provider will rely on these credentials in a variety of ways. Typically, the healthcare providers request information from these credentials for:
Scheduling—Healthcare providers rely on information from the patient's identification credential to locate that patient in the healthcare provider's electronic medical record systems (EMR), which are also referred to as electronic health record systems (EHR). The process of verifying a patient's identity is handled manually, and is often conducted over the phone or at the point of registration.
Pre-Registration—Prior to care, a healthcare provider generally contacts the patient by phone to verify that patient's unique eligibility relative to the healthcare benefits. The healthcare provider then uses the collected information to access that patient's detailed eligibility information via an existing communication channel with that benefit provider or with a medical claim clearinghouse.
Prior Authorization—Prior to care, a healthcare provider may establish if a given procedure requires prior authorization from a benefits provider.
Registration—On the day of care, a patient will check in at a physical location of the healthcare provider. At this point the provider will confirm both patient identity and patient eligibility, repeating the processes similar as those of the scheduling and pre-registration. The provider will also confirm patient coverage details specific to the care items to be implemented.
Medical Necessity—After care, the healthcare provider will initiate an insurance claim with the patient's insurance provider. Part of this claim process includes the insurance provider issuing medical guidelines, which provide clinical instructions to the healthcare provider to approve the claim.
Discharge or Referral—At the completion of care, the provider can initiate a referral to a specialist. At this point, the initial provider may begin the above process anew, by confirming the patient's eligibility and coverage similar to the Pre-Registration step.

### BRIEF DESCRIPTION OF THE SEVERAL VIEWS OF THE DRAWINGS

FIG. 1 is a network diagram showing a sample environment in which the facility operates in some embodiments.

FIG. 2 is a block diagram showing some of the components typically incorporated in at least some of the computer systems and other devices on which the facility operates.

FIG. 3 is a data flow diagram that shows interactions among distributed components of the facility according to some embodiments.

FIG. 4 illustrates an example architecture of self-sovereign identities according to some embodiments.

FIG. 5 shows an example system of various participants having private connection links according to some embodiments.

FIG. 6 is a flow diagram of an example process for issuing a credential according to some embodiments.

FIG. 7 is a flow diagram of an example process 700 for conducting a proof procedure on a credential according to some embodiments.

### DETAILED DESCRIPTION

The inventors have recognized that the current processes of handling credential in the health care field are not efficient and convenient for all the involved parties. For the patient, this process can amount to burdensome interactions with healthcare administrators prior to care, as well as a real barrier to care as many of these steps must be completed prior to non-emergent care. Additionally, because many of these processes are manual, and even the automated processes, such as electronic data interchange (EDI) transactions, rely on manually inputted data, errors at this point in a patient's care journey can lead to denials of insurance claims. Those difficulties put unnecessary emotional and financial stress on the patient after care, can lead to surprise bills, and leads to an overall challenge to patient understanding and planning for their healthcare finances.

The healthcare providers' challenges with the current system stem from the same challenges faced by the patient. First, because the provider must balance confirmation of coverage and eligibility leading up to care along with the logistics of patient identification and scheduling, real costs are incurred in the form of process, infrastructure, and resources to ensure that the patient's identity and coverage information is correct. Additionally, because of the manual processes involved, there is real downstream cost in the form of claims denials. Managing these denials is again the source of costs to the provider in terms of process and resource allocation, as well as in the delay of payment for care.

The insurance provider's challenges mirror those of the providers, as well as encompass a negative member experience. Because of the pains felt by the patient in this process, insurance providers are often blamed, resulting in a poor member experience. Additionally, the insurance provider suffers business impact from all of the process and cost due to maintaining the processes of information transaction, as well as costs due to manual errors and denials.

In response to recognizing these disadvantages, the inventors have conceived and reduced to practice a software and/or hardware facility for improved handling of health credentials. The facility generates verifiable and portable digital credentials for users or other participants, which can replace physical credentials and be automatically verified using encryption keys.

An issuer has the facility to issue a credential to a holder. For example, a hospital issuer may use the facility to issue a vaccination credential to a patient holder who was vaccinated by the hospital. A verifier uses the facility to verify a credential issued to a holder. For example, an airline carrier issuer may use the facility to verify a vaccination credential issued to a passenger holder.

A client of an issuer (“issuer client”) receives identification of a user, e.g., a patient, from a wallet applicant deployed on a user terminal of the user. Upon verifying the authenticity of the user identification information, the issuer client performs a data binding step by retrieving information of the user, e.g., medical record, from a data system of the issuer, e.g., an electronic medical record system, by mapping the verified user identification into the data system. The issuer client assembles the retrieved information into a credential template having a standardized data structure and format and provides the assembled credential information to the user.

In some embodiments, the issuer client establishes a trusted, private, and cryptographically secured connection with the wallet application of the user through an agent, e.g., a cloud-based agent, and provides the credential information to the wallet application via the private connection. For example, when the issuer client registers the wallet application with the cloud-based agent for the private connection, a pair of cryptographic keys “communication key pair” are generated or identified for the secured communication between the cloud-based agent and the wallet application. For example, the cloud-based agent encrypts the credential information using a public key of the communication key pair before sending the credential information to the wallet application. Upon receiving the encrypted credential information, the wallet application decrypts it using the private key of the communication key pair to obtain the credential information.

The issuer client digitally signs the credential so that the authenticity of the credential is verifiable by others. For example, the cloud-based agent digitally signs the credential using a private key of the issuer client and causes to store a corresponding public key of the issuer client on a distributed ledger that is accessible by other clients.

After the user receives the credential, the credential becomes portable for the user to the extent that the user can digitally present the credential to a client of a verifier. For example, a private, trusted, and cryptographically secured connection can be established between the wallet application of the user and the verifier client, through an agent. The verifier client can verify the authenticity of the credential information based on the digital signature of the issuer client, using the public key of the issuer client. In some embodiments, the signed credential contains a public identifier of the issuer client, which functions as storage identifier to locate the public key of the issuer client in the distributed ledger.

In order to access only a portion of the contents of the credential—such as vaccination date, to the exclusion of vaccine brand—with source and integrity verifications, the verifier client verifies the credential through zero knowledge proof because the verifier client only needs to verify, using the public key of the issuer client, that the digital signature attached on the credential information is authentic and belongs to the issuer client, thereby proving that the credential information has not been tampered since it is generated by the issuer client. Such tamper-evident credential is more trustworthy than the physical credentials and is more convenient for the user to maintain and present the credential information to various verifiers. The digital credential is also more portable. Verifiers only need to access to the public ledger to obtain the public key to verify the digital credential and they do not need to maintain complex infrastructure with issuing party or clearinghouses.

The facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, the public identity ledger stores the public DIDs of participants in a decentralized manner, which enables participants to access DIDs of other participants in a reliable manner. Such public identity ledger substantially reduces the interactions and thus the computing resources and communication bandwidth needed for participants to establish private connections because the publicly accessible public DIDs provide the information for establishing the private connections between any two participants. The use of decentralized ledger also enables that the records stored thereon cannot be modified or tampered without being detectable by other nodes maintaining the ledger, which improves data security.

The facility enables all users to reduce or eliminate hardware equipment for maintaining, handling or reading physical credentials and the interactions involved in verifying the physical credentials. For example, a healthcare provider does not need to maintain as many scanners or copy machines to copy driver's licenses and insurance cards of patients because patients present such credentials digitally. Healthcare providers and insurances companies do not need to conduct a great amount of phone calls back and forth because the insurance coverage of patients can be verified digitally through the signed credentials.

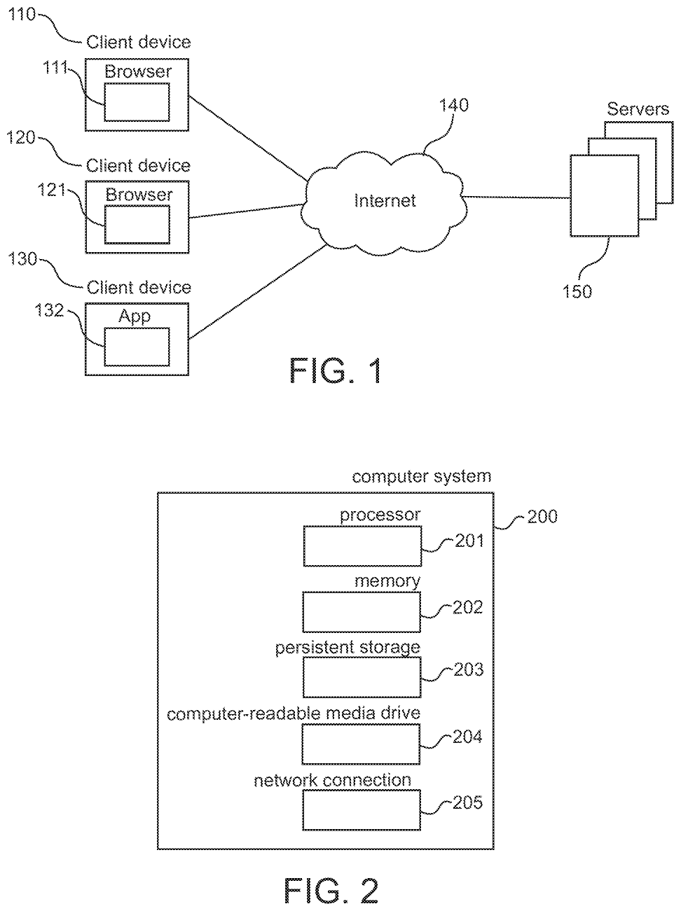

FIG. 1 is a network diagram showing a sample environment in which the facility operates in some embodiments. A number of client devices 110, 120, and 130 are connected via the Internet 140 or another network to one or more servers 150 that operate a cloud-based platform. Some of the client devices—such as client device 110, 120—execute a browser 111, 121 that interacts with the cloud-based platform software on the server on behalf of a user of the facility using the client device, while other client devices—such as client device 130—execute a specialized mobile application or desktop application 132 that interacts with the cloud-based platform's software on the server on behalf of a user using the client device.

FIG. 2 is a block diagram showing some of the components typically incorporated in at least some of the computer systems and other devices on which the facility operates, including the devices shown in FIG. 1. In various embodiments, these computer systems and other devices 200 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 201 for executing computer programs and/or training or applying machine learning models, such as a CPU, GPU, TPU, NNP, FPGA, or ASIC; a computer memory 202 for storing programs and data while they are being used, including the facility and associated data, an operating system including a kernel, and device drivers; a persistent storage device 203, such as a hard drive or flash drive for persistently storing programs and data; a computer-readable media drive 204, such as a floppy, CD-ROM, or DVD drive, for reading programs and data stored on a computer-readable medium; and a network connection 205 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

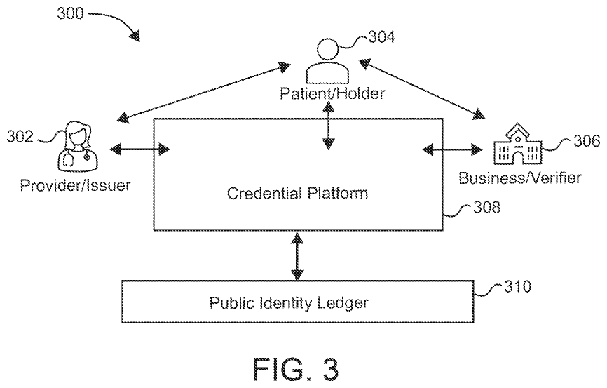

FIG. 3 is a data flow diagram that shows interactions among distributed components of the facility in some embodiments. The diagram 300 includes one or more providers or issuers 302 (issuer 302) that issue and provide credentials to one or more patients or holders 304 (patient 304) that receive and hold the credentials. The credentials received at the patients 304 become portable and are ready for the patients 304 to present them to one or more business users or verifiers 306 (verifier 306) that verify the credentials. In some embodiments, the issuers 302, the patients 304 and the verifiers 306 each interact with agents established in a credential platform 308, for example, a platform of Evernym, in handling the credentials. For example, the issuers 302 send the credentials through agents in the credential platform 308 to the patients 304. The patients 304 present the credentials through agents in the credential platform 308 to the verifiers 306. In some embodiments, the issuer 302, through agents in the credential platform 308, attaches a digital signature to a credential using a private key of the issuer 302 before sending the credential to the patient 304. The respective agents in the credential platform 308 also causes to store a public key of the issuer 302 in a public identity ledger 310, e.g., a distributed ledger or a blockchain. The verifier 306 receives the signed credentials, obtains the public key of the issuer 302, and verifies the authenticity of the digital signature using the public key of the issuer 302, all through the respective agent in the credential platform 308.

In some embodiments, the issuers 302, patients 304 and verifiers 306 interact with one another without credential platform. For example, the issuers 302 writes, e.g., through a local agent, a public DID directly to the public identity ledger 310, and the verifier 306 reads, e.g., through a local agent, the public DID of the issuer 302 directly from the public identity ledger 310. The patients 304 can establishes private connections with issuers 302 and/or verifiers 306 directly with the credential platform 308, with or without local agents thereof.

Besides the communications through agents in the credential platform 308 with respect to the credentials, the issuer 302 and the verifier 306 each also have communication links with the patient 304, respectively. In some embodiments, the use of the agents in the credential platform 308 is reserved for actions that are directly related to the handling of credentials. Peripheral activities that are not directly related to the handling of credentials are conducted through the one-to-one communication link between the issuer 302 and the patient 304 or between the patient 304 and the verifier 306. The issuer 302 and verifier 306 are defined only with respect to a specific credential. An issuer 302 with respect to a first credential may be a verifier with respect to a second credential. Similarly, a verifier 306 with respect to a first credential may be an issuer 302 with respect to a second credential.

With the signed credentials, the patient 304 becomes the source of their credential information, e.g., personal identity, insurance policy coverage, health record, vaccination record, etc. With the credentials digitally signed by the private key of the issuer 302 and the corresponding public key stored in the distributed identity ledger 310, the verifier 306 has the capacity to verify and trust the credentials presented by the patient 304 without contacting the issuer 302 or third-party clearinghouses and other middleman services.

The public identity ledger 310 provides a means for any number of participants to publish and access a public key or a public decentralized identifier “public DID” in the public identity ledger 310. In some embodiments, the identity ledger 310 is not centrally managed. Instead, participants on the network establish an agreed-upon method of consensus. With this consensus, and with cryptographic methodologies that ensure the immutability of past transactions, all participants on the network can typically trust the information stored in the public identity ledger 310, regardless of their trust relative to any one of other network participants. A centrally managed identity ledger is also possible and included in the disclosure.

Specifically, issuers 302 and verifiers 306 have the means, e.g., through the credential platform 308, to add standardized digital identity markers, referred to as decentralized identifiers or DIDs, to the identity ledger 310, making those decentralized identifiers immutable and secure. These decentralized identifiers contain information on where and how to communicate with the issuers 302, verifiers 306 as identity owners, also referred to as “digital endpoints”, as well as public cryptographic keys to communicate with the identity owners. With this information, any network participant can establish a secondary, unique and private line of communication that inherits all of the security and immutability of the participants' decentralized identifiers on the public identity ledger. In the healthcare space, such a ledger can exist independently from a specific healthcare provider or insurance provider, either through existing, public blockchain networks like the Sovrin network, or as consortium-governed healthcare-specific blockchain networks. With such a blockchain network in place, healthcare providers (as issuers 302 and/or verifiers 306), and insurance providers (as issuers 302 and/or verifiers 306), and in some embodiments, even patients (as patients 304) can all act as participants on the network, registering their own unique, secure, and immutable public decentralized identifiers. A participant on the network of the public identity ledger 310 does not necessarily function or have the authority to function as a mining node or as a consensus node to maintain a copy of the public identity ledger and to add a public DID to the public identity ledger 310. Further, a participant does not necessarily interact with the public identity ledger 310 directly. In some embodiments, the issuer 302, verifier 306 and patient 304 each interacts with the identity ledger 310 through respective agents (not shown in FIG. 3) registered on the credential platform 308.

In some embodiments, a patient 304 or other holders 304 of a credential do not store public DIDs in the public identity ledger 310. Instead, a patient 304 establishes an initial connection with an issuer 302 or a verifier 306 through other existing applications or mechanisms of the issuer 302 or the verifier 306. For example, a patient 304 may establish an initial connection with a hospital issuer 302 through a portal terminal of the hospital issuer 302.

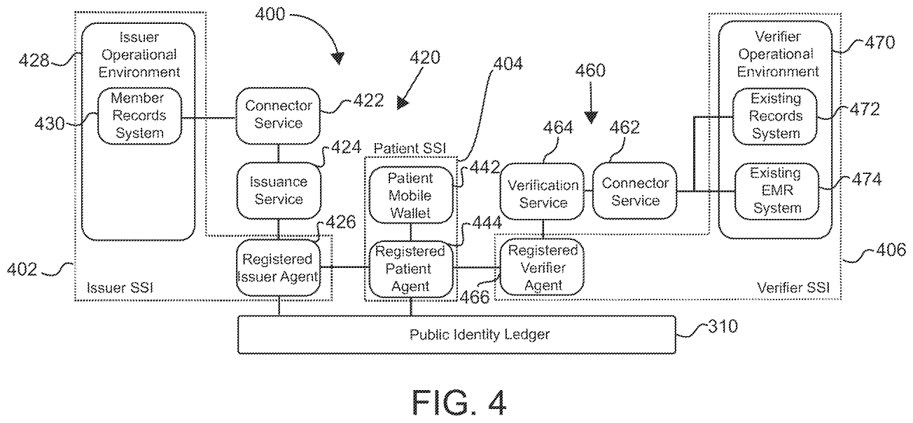

FIG. 4 illustrates an example architecture 400 of self-sovereign identities of the issuers 302, patients 304 and verifiers 306 of FIG. 3. Referring to FIG. 4, a self-sovereign identity (SSI) 402 of an issuer 302 includes a registered issuer agent 426 coupled to an issuer operational environment 428 that includes a member records system 430. A self-sovereign identity 406 of a verifier 306 includes a registered verifier agent 466 coupled to a verifier operational environment 470 that includes an existing records system 472 and an existing electronic medicine records “EMR” system 474. A self-sovereign identity 404 of a patient 304 includes a patient mobile wallet 442 and a registered patient agent 444. An agent 422, 444, 466 is an application or service that itself is registered on the identity network 310 and is identified as within the scope of the distributed identity of a participant (an issuer 302, a patient 304, or a verifier 306). An agent can be a local service on a participant's terminal device, also referred to as an “edge agent”, or a cloud-based service that is able to interact with other agents at all times, referred to as a “cloud agent”. A participant can have more than one agent as part of its self-sovereign identity SSI for interacting with other participants of the diagram 300.

In some embodiments, the registered issuer agent 426 and the registered verifier agent 466 are cloud agents set up on the credential platform 308. The registered patient agent 444 is an edge agent or a cloud agent. In a case where the registered patient agent 444 is an edge agent, it is part of the patient mobile wallet 442 or functions together with the patient mobile wallet 442.

Referring to FIGS. 3 and 4 together, in addition to the communications through the public DIDs, participants of the diagram 300 also establish trusted, private, and cryptographically secured lines of communication between any two participants. An issuer 302 can establish unique connections with various patients 304 through respective connection requests, and a verifier 306 can establish unique connections with patients 304 on an as-needed basis. Initial connection requests can be made using the public DID information of the target participant to be connected, which is stored on the public identity ledger 310. This public DID document defines the endpoint at which the participant can be reached, as well as details on how to establish a secured connection, for example, by using the public key of the target participant. Upon an initial connection being established through the public DID, the two participants can then proceed to establish their private DIDs dedicated or unique for the private secured connections therebetween, which include private endpoints and key pairs for the private connections. The private DIDs are stored in the respective self-sovereign identities SSIs, e.g., the patient mobile wallet 442 and the agents 426, 444, 466 of FIG. 4, and not in the public identity ledger 310, making the connections and all traffic on the connections hidden, private, and secure.

In some embodiments, issuer 302, patient 304, and verifier 306 exchange digital credentials through the trusted, private, cryptographically secured connections established under the private DIDs through agents 426, 444, 466 (or wallet 442 when the wallet 442 includes the functions of the patient agent 444). An issuer 302 includes an issuer service 420 that includes a connector service 422 and a credential issuance service 424. The credential issuance service 424 is coupled to the existing information database 430, e.g., a member records system 430, of issuer 302 to create a digital credential using information retrieved from the existing database 430. In some embodiments, the credential issuance service 424 is coupled to the existing information database 430 via the connector service 422, e.g., an application programming interface. The issuance service 424 selects a credential template including standardized data structure and format, and populates the credential template with the information retrieved from the existing database 430 to generate the digital credential. The issuer service 420 communicates the digital credential to the respective patient 304 by means of agents 426, 444 of the respective self-sovereign identities 402, 404 using the trusted, private, cryptographically secured connections. The credential may also be based on data of other sources, e.g., manually inputted data through a portal terminal or online portal page.

A digital credential is a list of claims specific to a patient 304. A claim is a piece of information attested to by the issuer 302. A simple example of a claim in this scenario can be the patient's member ID stored in the member records system 430. A more complicated claim can be the entirety of the eligibility information of the patient 304. The credential contain claims that encompass that patient's full coverage, eligibility, prior authorization, and medical guidelines information, as well as her/his personal information such as name and member ID. Depending on the issuer 302 and the issuer's local database 430, the credential can also contain the medical or health record information of a patient 304 or other information.

In some embodiments, the issuer agent 426 of the SSI 402 of the issuer 302 maintains a private key of the issuer 302 and digitally signs the credential before sending the credential to the patient agent 444 of the SSI 404 of the patient 304.

In some embodiments, when patient 304 receives the credential, the patient agent 444, or the wallet 442 when wallet 442 incorporates the functions of the agent 444, signs the received credential with two pieces of information: a blinded link secret and a blinded proving secret unique to the patient 304. The blinded proving secret provides a means for a verifier 306 to verify that the credential belongs to the patient 304. The blinded link secret enables that credentials containing the same blinded link secrets can be combined. The attached secret information of the patient 304, along with the cryptographic signature of the issuer 302 on the credential, provides a secure means to verify that the issuer 302 issues the credential to the patient 304.

The wallet 442 of the SSI 404 of the patient 304 stores the credentials. The wallet 442 is an application that resides in the terminal device of patient 304 and is part of the self-sovereign identity 404 of the patient 304.

The self-sovereign identity 404 of the patient 304 can present the credential to the self-sovereign identity 406 of the verifier 306 through the trusted, private and cryptographically secured connections between the patient agent 444 of the self-sovereign identity 404 of patient 304 and verifier agent 466 of the self-sovereign identity 406 of verifier 306. Any time a patient 304 provides information from one or more of her/his credentials to a verifier 306 on the network, the patient 304 does so through the respective wallet 442 using a procedure referred to as a “proof procedure.” Generally, a proof procedure begins with a proof request from a verifier 306 that requests for one or more pieces of information contained in the credentials of the patient 304. For example, the proof request digitally asks the patient 304 for cryptographically robust evidence for the one or more pieces of information. For example, the verifier 306 can make a proof request for the patient's eligibility status for a specific Current Procedural Terminology (CPT) code. The proof request also asks for a cryptographic proof, e.g., a storage identifier for a public key of the issuer 302 or the patient 304, which the verifier agent 466 can use to obtain the public key to verify the digital signatures on the credential.

Upon receiving the credential from the patient agent 444 of the patient 304, the verifier agent 466 of the verifier 306 verifies the credential as belonging to the patient 304 and as being issued by issuer 302. In some embodiments, the verification includes the verifier agent 466 obtaining from the public identity ledger 310 the public keys of one or more of the issuer 302 or the patient 304. The verifier agent 466 of the self-sovereign identity 406 of the verifier 306 uses the obtained public keys to read or verify the digital signatures that the issuer agent 426 of the issuer 302 or the patient agent 444 of the patient 304 have attached to the credential. Upon successful verification of the credential, the verifier agent 466 of the verifier 306 communicates with the verifier service 460, e.g., including the verification service 464 and the connector service 462, to route the information contained in the credential into the verifier's local database system, e.g., the existing records system 472 or the existing EMR system 474.

In the verification of the received credential, the verifier agent 466 of the verifier 306 also determines whether the issuer 302 has revoked the credential. In some embodiments, a proof procedure does not establish a direct line of information flow between the self-sovereign identity 402 of the issuer 302 and the self-sovereign identity 406 of the verifier 306. However, the issuer 302 has the authority to revoke a credential that it has issued to a patient 304. For example, the issuer 302 maintains a revocation list on the public identity ledger 310. By checking the revocation list, the verifier agent 466 of the verifier 306 determines whether the credential under verification has been added to the revocation list, i.e., has been revoked by the issuer 302, and is thus invalid.

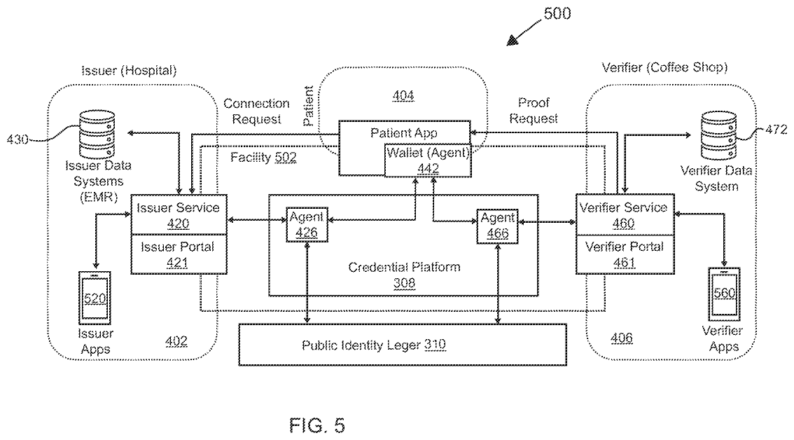

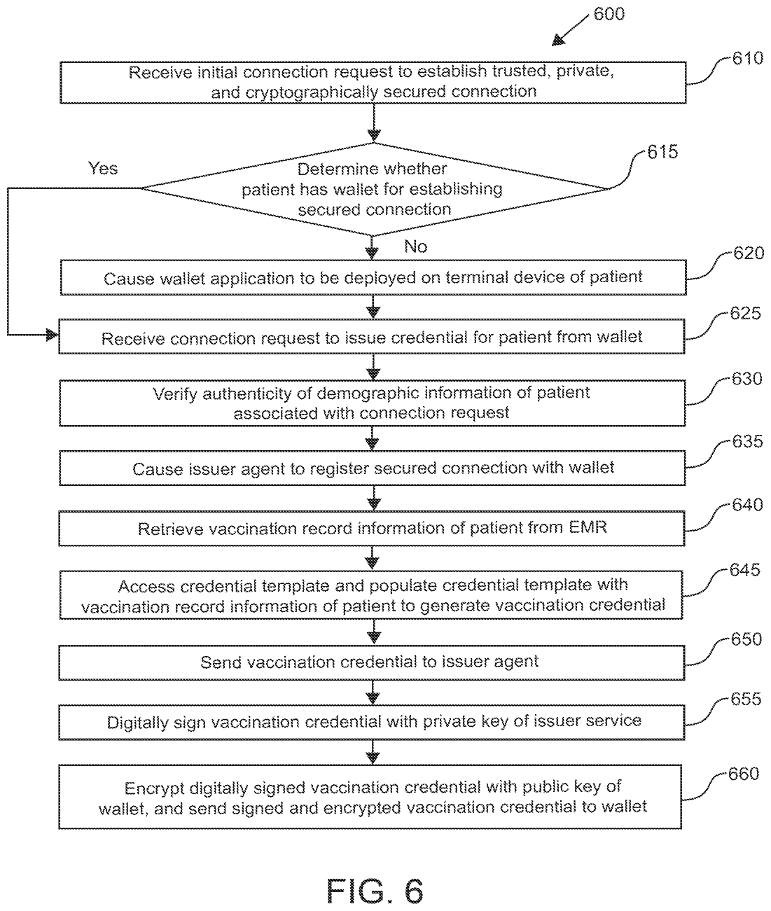

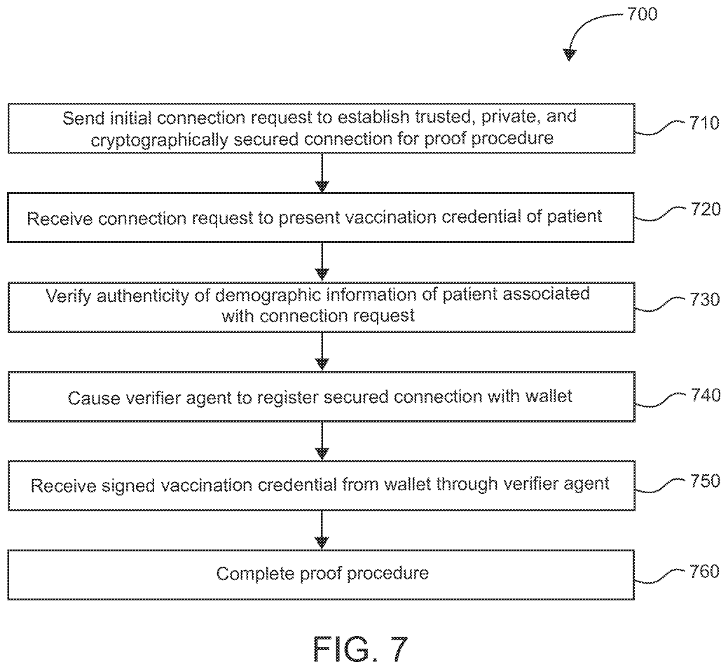

FIG. 5 shows an example system 500 of various participants having private connection links. FIG. 6 is a flow diagram of an example process 600 for issuing a credential to a patient. FIG. 7 is a flow diagram of an example process 700 for conducting a proof procedure on the credential issued to the patient. In the description herein, a vaccination record of a patient 304 is used as an example to illustrate the processes 600, 700.

Referring to FIGS. 5 and 6 together, a facility 502 provides the overall solutions and executable program codes or software applications to implement each of the components of the diagram/system 300, 400 of FIGS. 3 and 4. For example, the facility 502 includes program codes or applications for implementing an issuer service 420 and an issuer portal 421 of a self-sovereign identity 402 of an issuer 302, a patient application including wallet 442 of a self-sovereign identity 404 of a patient 304, and a verifier service 460 and a verifier portal 461 of a self-sovereign identity 406 of a verifier 306. As an illustrative example, the issuer 302 is an example hospital, the verifier 306 is an example coffee shop, and the patient 304 has taken a vaccination at the hospital 302, the record of which is stored on the data system 430 of the issuer 302. The patient 304 visits the coffee shop 306, which requires a customer to show vaccination record before being catered.

In example operation 610, the patient 304 initiates and the issuer service 420 receives an initial connection request to establish a trusted, private, and cryptographically secured connection. The initial connection request can be communicated to the issuer service 420 through any means, which are all included in the scope of the disclosure. For example, an existing issuer application 520 of the hospital 302 receives the initial connection request of the patient 304 through, e.g., a patient registration system of the hospital 302; the issuer portal 421 receives the initial connection request of the patient 304; or the issuer service 420 receives the initial connection request of the patient 304 directly from the wallet 442 of patient 304. In some embodiments, the issuer portal 421 includes one or more of a portal terminal, e.g., positioned physically in the hospital 302, or an online portal having a secure login for a patient 304 to access. The issuer portal 421 can present a two-dimensional code, e.g., a quick response “QR” code, either through a display of the portal terminal or through online graphic presentation, for the patient 304 to scan so as to submit the initial connection request.

The initial connection request includes or is associated with some basic identification information of patient 304, including one or more of patient name, address, phone number, or other identification number, for the hospital 302 to locate the vaccination record of the patient 304.

In some embodiments, the initial connection request is not communicated through the credential platform 308. The initial connection request is communicated between the hospital 302 and the patient 304 based on the information provided by the DIDs of the hospital 302 and identity information of the patient 304 provided to the hospital in various means. In some embodiments, patients 304 with identity credentials of their identity can use those identity credentials directly with a hospital. With respect to the identity credentials, the hospital functions as a verifier 306 and verifies the identity credential. Upon successful verification of the identity credential, hospital 302 uses the identity information contained in the identity credential for the initial connection request.

In some embodiments, the initial connection request also includes or is associated with an identifier of a terminal device of the patient 304, for example, a cell phone number, a hardware identifier, a UID, a network identifier or other identifier of a terminal device of the patient 304.

In example operation 615, issuer service 420 determines whether the patient 304 has a wallet 442 for establishing the secured connection. In a case where patient 304 already has a wallet 442 set up, the patient 304 can identify the wallet 442 in the initial connection request, e.g., through the DID stored in the public identity ledger 310. The historical transaction data between the hospital 302 and the patient 304 also indicates whether a wallet 442 of the patient 304 has been used in a secured connection or other transactions with the hospital 302. In some embodiments, once established, a private connection can be stored and re-used between issuer 302 and patient 304.

In response to that the issuer service 420 determines that the patient 304 has a wallet 442, the process jumps to operation 625. If there is an existing connection to a wallet 442, then the [process can jump to operation 640.

In example operation 620, in response to that issuer service 420, or a client application 520 coupled to the issuer service 420, determines that patient 304 does not have a wallet 442 for establishing a secured connection, the issuer service 420 causes a wallet application 442 be deployed on a terminal device of the patient 304. For example, the issuer portal 421 provides information about where and how the patient 304 can download or otherwise access the programs to deploy a wallet application on the terminal device of the patient 304. For another example, the issuer service 420 sends a message to the patient 304, e.g., an SMS message, an email message, or other types of messages based on the identifier of the patient 304 or the identifier of the terminal device of the patient 304, which includes a link to the programs for deploying a wallet. The patient 304 can download the program through the link embedded in the message.

In some embodiments, the issuer service 420 automatically sends the message with wallet program link to the patient 304 without determining whether the patient 304 has a wallet 442 in the patient's self-sovereign identity 404. Then the patient 304 has the option not to download the wallet program if the patient 304 has an existing wallet 442.

In example operation 625, the wallet 442 of the patient 304 sends, and the issuer service 420 receives (it is also possible that the issuer service 420 sends and the wallet 442 of the patient 304 receives), a connection request to issue a credential for the vaccination record of the patient 304. The connection request includes demographic information of the patient 304 and the private DID information of wallet 442 dedicated for a trusted, private, cryptographically secured connection between the self-sovereign identity 402 of the hospital 302 and the self-sovereign identity 404 of the patient 304. Specifically, the private DID is dedicated for private connection between the issuer agent 426 on the credential platform 308 and the wallet 442. The demographic information of the patient 304 enables the issuer service 420 to match the demographic information with the patient record in the EMR database 430 of the hospital 302. The private DID information includes the endpoint information and a public key for the private connection between the self-sovereign identity 402 and the self-sovereign identity 404. This private DID is different from the public DID of the patient 304, if any, that is stored in the public identity ledger 310. Note that the wallet 442 keeps the private key of this private DID, which the wallet 442 uses to decrypt a communication of the credential encrypted using the public key of the private DID.

In some embodiment, the wallet 442 is configured to identify the issuer service 420 as corresponding to the issuer 302. For example, the wallet 442 is configured to provide or display a list of issuers 302 for the patient 304 to select the hospital 302 for the vaccination credential. The wallet 442 then identifies the issuer service 420 as corresponding to the hospital 302 based on the patient's selection of the hospital 302.

In example operation 630, the issuer service 420 verifies the authenticity of the demographic information of the patient 304 contained in or associated with the connection request with the patient information stored in the EMR system 430 or other database that the hospital 302 accesses.

In example operation 635, the issuer service 420 causes the issuer agent 426 of the self-sovereign identity 404 of the hospital 302 to register a secured connection with the wallet 442. In some embodiments, the issuer service 420 provides the private DID of the wallet 442 to the issuer agent 426 of the SSI 402 of the hospital 302. The issuer agent 426 stores the public key of the private DID of the wallet 442 and uses the private endpoint to establish the private connection with the wallet 442.

In some embodiments, the issuer service 420 establishes a new cloud-based agent 426 for the private connection with the wallet 442 on the credential platform 308 that is remote from both the issuer service 420 and the wallet 442. In some embodiments, the cloud-based agent 426 is existing as an agent for the issuer service 420 in the self-sovereign identity 402 and issuer service 420 registers the private connection with the wallet 442 with the existing cloud-based agent 426.

In some embodiments, a connection request contains a request to issue the vaccination credential. In some embodiments, the connection request and the request to issue the vaccination credential are two separate requests received by the issuer service 420 from the wallet 442. In some embodiments, the request to issue the vaccination credential also identifies a credential template for the vaccination credential. The credential template includes, among others, standardized data structure and format for the credential.

In some embodiments, in registering the secured connection with the wallet 442, the issuer agent 426 creates a key pair of a public DID of the issuer service 420 for signing a credential to be sent to the wallet 442. The issuer agent 426 stores the private key of the public DID and causes the public key of the public DID to be published in the public identity ledger 310.

In example operation 640, the issuer service 420 retrieves the vaccination record information of the patient 304 from the EMR 430 in the self-sovereign identity 402 of the hospital 302 by matching the demographic information of the patient 304 with the EMR 430. In some embodiments, the retrieval of the vaccination record information of the patient 304 is conducted in a manner under a standard healthcare application programming interface, e.g., the HL7 standardized interfaces including the Fast Healthcare Interoperability Resource FHIR protocol. For example, the retrieved vaccination record information of the patient 304 has a standardized information content and structure under the FHIR protocol.

In example operation 645, the issuer service 420 generates the vaccination credential using the vaccination record information of the patient 304. In some embodiments, the issuer service 420 selects or accesses a credential template with standardized data structure and format for the credential. A standardized data structure and format of the credential enables the credentials issued by different issuers 302 to be compatible with one another and makes the verification of the credentials more efficient. The issuer service 420 populates the standardized credential template with the vaccination record information of the patient 304 to generate a vaccination credential. In some embodiments, the standardized credential template is registered on the public identity ledger 310.

In example operation 650, the issuer service 420 sends the vaccination credential to the cloud-based issuer agent 426.

In example operation 655, the issuer agent 426 digitally signs the vaccination credential with the private key of the public DID of the issuer service 420. In some embodiments, according to an agent policy registry of the self-sovereign identity 402, the issuer agent 426 stores the private key of the issuer service 420. The issuer agent 426 also causes a public key of the issuer service 420 to be stored in the public identity ledger 310. The public key corresponds to the private key used to sign the vaccination credential and is part of the public DID of the hospital 302, which other participants of the diagram 300 can access to verify the authenticity of the digital signature attached to the vaccination credential issued by the issuer service 420.

In example operation 660, the issuer agent 426 encrypts the digitally signed vaccination credential with the public key of the private DID of the wallet 442, and sends the signed and encrypted vaccination credential to the private endpoint defined by the private DID of the wallet 442.

Those skilled in the art will appreciate that the acts shown in FIG. 6 and in each of the flow diagrams discussed herein may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

Upon receiving the signed and encrypted vaccination credential, the wallet 442 decrypts it using the private key of the private DID to obtain the signed vaccination credential. In some embodiments, wallet 442 chooses to further verify the digital signature of the signed vaccination credential using the public key of the issuer service 420 as part of the public DID of the issuer service 420 stored in the public identity ledger 310. In some embodiments, the wallet 442 further digitally signs the signed vaccination credential by attaching one or more of a blinded link secret and a blinded proving secret unique to the patient 304 to the signed vaccination credential. The wallet 442 maintains the signed vaccination credential as a piece of portable credential that the patient 304 can present to a verifier 306 in a proof procedure.

Referring to FIGS. 5 and 7 together, the process 700 of a proof procedure starts with example operation 710, where the verifier service 460 of the coffee shop 306 sends, and the wallet 442 of the patient 304 receives, an initial connection request to establish a trusted, private, and cryptographically secured connection for the proof procedure. The initial connection request is triggered when the patient 304 visits the coffee shop 306 and interacts with the existing application 560 of the coffee shop 306. Coffee shop 306 requires that a customer present proof of vaccination before being catered at the coffee shop 306. In the initial connection request, the verifier service 460 is able to identify the wallet 442 of the patient 304 based on the identification information of the patient 304 that the coffee shop application 560 collects or the verifier portal 461 receives through inputs by the patient 304. In some embodiments, one or more of the verifier service 460 or the wallet 442 retrieves the public DID of the other party in the initial connection request.

Note that in the description of the process 700, it is assumed that the patient 304 already has a wallet application 442 in the self-sovereign identity 404 and has a vaccination credential stored thereon. In a case where a patient 304 does not have a wallet established on her/his terminal device, the verifier service 460 can cause a wallet application be deployed on the terminal device of the patient 304 following similar operations like operations 615 and 620 described herein.

The initial connection request can be communicated between the verifier service 460 and the wallet 442 through any means, which are all included in the scope of the disclosure. For example, the verifier portal 461 presents a two-dimensional code, e.g., a quick response “QR” code, either through a display of a portal terminal or through an online graphic presentation for the patient 304 to scan so as to communicate the initial connection request.

In example operation 720, the wallet 442 of the patient 304 sends, and the verifier service 460 receives, a connection request to present a vaccination credential of the patient 304. The connection request includes demographic information of the patient 304 and private DID information of wallet 442 dedicated to a trusted, private, cryptographically secured connection between the self-sovereign identity 406 of the coffee shop 306 and the self-sovereign identity 404 of the patient 304, or specifically between the verifier agent 466 of the self-sovereign identity 406 and the wallet 442 of the self-sovereign identity 404. The demographic information of the patient 304 enables the verifier service 460 to match the demographic information with the customer record in the data system 472 of the coffee shop 306. The private DID information includes private endpoint information and a public key of the private DID for the private connection between the self-sovereign identity 406 and the self-sovereign identity 404. This private DID is different from the public DID of the patient 304, if any, that is stored in the public identity ledger 310. The wallet 442 keeps the private key of the private DID dedicated for the private connection with the self-sovereign identity 406, which the wallet 442 uses to encrypt a communication of the vaccination credential to be presented to the verifier service 460. It should be noted that the private DID of the self-sovereign identity 404 of the patient 304 for the private connection with the self-sovereign identity 406 of the coffee shop 306 is different from the private DID for the private connection with the self-sovereign identity 402 of the hospital 302.

In some embodiments, in the connection request, the wallet 442 presents an identification credential containing the demographic information of the patient 304. This identification credential can come from a number of sources, including the government as a participant of the diagram 300 or an issuer 302. For example, the issuer 302 can issue an identification credential after verifying the authenticity of the demographic information of the patient 304 in a similar way as issuing the vaccination credential as described in the process 600 of FIG. 6. The identification credential can be verified by the verifier service 460 in a similar way as verifying a vaccination credential as described herein.

In example operation 730, the verifier service 460 verifies the authenticity of the demographic information of patient 304 contained in the connection request with the customer information stored in the coffee shop data system 472. In some embodiments, the verifier service 460 verifies an identification credential of the patient 304.

In example operation 740, the issuer service 420 causes the verifier agent 466 of the self-sovereign identity 406 of the coffee shop 306 to register a secured connection with the wallet 442, by providing the private DID of the wallet 442 to the verifier agent 466. The verifier agent 466 stores the public key of the private DID of the wallet 442 and uses the private endpoint of the private DID to establish a private connection with the wallet 442.

Upon the private connection being established, the wallet 442 is able to identify the verifier agent 466 of the self-sovereign identity 406 as coupled to the verifier service 460.

In example operation 750, the verifier service 460 receives signed vaccination credential from the wallet 442 through the verifier agent 466 in the secured connection. Specifically, the wallet 442 encrypts the signed vaccination credential with the private key of the private DID dedicated for the private connection with the verifier agent 466. The signed vaccination credential includes digital signature of the issuer service 420 and the digital signature of the wallet 442, e.g., one or more of the blinded link secret and blinded proving secret unique to the patient 304. Upon receiving the signed and encrypted vaccination credential, the verifier agent 466 decrypts the signed and encrypted vaccination credential using the public key of the private DID of the wallet 442 to obtain the signed vaccination credential. The verifier agent 466 verifies the authenticity of the digital signatures attached to the vaccination credential using public keys of the issuer service 420 and the wallet 442 retrieved from the public identity ledger 310. The verifier agent 466 also determines whether the vaccination credential is listed in a revocation list of the issuer service 420 stored on the public identity ledger 310.

In a case where multiple credentials are presented by the wallet 442, e.g., a vaccination credential and an identification credential, the verifier agent 466 determines, through the blinded link secret of each of the credentials obtained using the public key of the public DID of the patient 304, that the multiple credentials are linked to one another and all belong to the same patient 304.

In example operation 760, the verifier service 460 completes the proof procedure. The vaccination credential or the proof result of the vaccination credential, is added to the data system 472 of the coffee shop 306 and the result of the proof procedure is communicated to the verifier application 560 thereby enabling the patient 304 to receive catering service at the coffee shop 306.

It should be noted that the processes 600, 700 are described in a way to facilitate understanding of the techniques, which does not mean to limit the scope of the disclosure. For example, the communications of data in the processes 600, 700, do not necessarily follow the directions of the communication, e.g., the sending or the receiving of information, as described herein. For example, the connection request of the process 600 can be achieved by the issuer service 420 sending the private DID of the issuer service 420 to the wallet 442. The proof request of the process 700 can be achieved by the verifier service 460 sending the private DID of the verifier service 460 to the wallet 442. The self-sovereign identity 404 of the patient 304 can have a cloud-based agent 444 that operates all the time and is coupled to the wallet 442 in a similar manner as those between the issuer service 420 and the issuer agent 426 or as those between the verifier service 460 and the verifier agent 466.

Further, although a patient is used as an illustrative example of a holder of a credential, a holder 304 may be other type of individual users or entity users, which are all included in the scope of the disclosure.

By performing in some or all of the ways described above, the facility 502 substantially improves the efficiency in the healthcare industry in handling patient credential information. By creating private, trusted, and cryptographically secured connections with insurance providers and with healthcare providers, patients can thus present their information in a portable, trustable and verifiable way as desired herein. Pre-registration, prior authorization, identification, and even day-of registration can be replaced by a simple, digital interaction between the healthcare providers and the patients. For example, in the weeks before care, a patient can receive a notification that a provider is requesting specific identity and coverage information. By accepting that request, the patient supplies the provider with the detailed information the provider needs to confirm the patient identity, establish eligibility, access coverage details, and gain prior authorization if needed. On the day of care the patient can receive a notification upon arrival through the wallet application, or scan a QR code at the portal application at the registration desk establishing secured connections for presenting the credential information of the patient. All of these transactions present the needed data to the healthcare provider in a way that is trustworthy, secure, and convenient. Further, the digital nature of the credential enables the unlimited amount of information contained in a credential as compared to the limited information represented by a physical credential nowadays.

This all highlights the simplest transaction a patient can allow using their credentials. However, this facility 502 enables much more complex and useful interactions as well. Important to the healthcare space is the concept of Protected Health Information (PHI). The management and control of PHI is central to current healthcare systems (e.g., EDI X12), and a system utilizing digital credentials increases the ability for the patient to control their own PHI. One way in which this is done is though zero-knowledge proofs. For example, a provider can require that a patient prove that they have a current, valid health benefits plan. Today, the patient would meet that request by providing their demographic information (driver license) and coverage information (access to their benefits information via their insurance provider) in full. This volume of data transferred for one specific request is one driving factor in the current regulatory environment. However, with digital credentials, the patient can prove they have a current, valid health benefit plan without even revealing the name of that plan and yet providing it in a way that is cryptographically trustable by the verifier and cannot be counterfeit. With a zero-knowledge proof, patients can combine claims within their credential into new information logically derived from one or more claims and present them in a cryptographically robust way to satisfy specific proof requests without revealing the underlying claims used to validate that proof.

Another technical advantage is the patient's ability to satisfy proof requests using multiple credentials issued by one or more issuers via a concept called compound proofs. Imagine the provider needs to not only get proof that the patient has a current, valid plan, but that the plan can be assigned to an existing patient record in their EMR. In this case the provider requires a predicate that itself relies on both coverage and demographic information. A compound proof behaves the same as the proofs described above, but utilizes claims from multiple credentials. The proof itself includes the patient's blinded link secret. This link secret provides a cryptographic means for the provider to verify that the multiple credentials used to satisfy the proof can be combined together. In this scenario, the predicate combines plan information from the coverage record with an EMR record number from the patient's demographic credential to satisfy the proof request.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
by a first client deployed on a terminal device of a first service provider:
receiving a request for secure connection from a wallet client deployed on a terminal device of a user, the request for secure connection including a first digital identifier of the wallet client, a first public key of the wallet client, and first personal identification information of the user;
in response to the request for secure connection:
verifying that the first personal identification information of the user is authentic;
in response to verifying that the first personal identification information of the user is authentic, sending the digital identifier of the wallet client and the first public key of the wallet client to a first cloud-based agent to enable the first cloud-based agent to store the digital identifier of the wallet client together with the first public key of the wallet client;
receiving a request to issue a credential that identifies a credential template; and
in response to receiving the request to issue a credential:
accessing the identified credential template;
retrieving information of the user from a first electronic health record system coupled to the first client;
populating the accessed credential template using the information of the user retrieved from the first electronic health record system to generate first credential information; and
sending the first credential information with the wallet client's digital identifier to the first cloud-based agent to enable the first cloud-based agent to:
encrypt the first credential information with the first public key of the wallet client; and
forward the encrypted first credential information to the wallet client using the wallet client's digital identifier.

2. The method of claim 1 wherein the retrieving information of the user from the first electronic health record system is performed in accordance with a standard health care application programming interface.

3. The method of claim 1, wherein the receiving the request for secure connection from the wallet client includes receiving the request for secure connection through the wallet client capturing a two-dimensional barcode displayed by a portal application of the first client, and receiving a phone number of the user.

4. The method of claim 1, further comprising:
by the first client:
invoking the first cloud-based agent to register the first service provider, to enable the first cloud-based agent to:
create a second key pair for the first service provider comprising a second private key and second public key;
store the second private key; and
cause publication of the second public key in a distributed ledger, and wherein the sending the first credential information with the wallet client's digital identifier further enables the first cloud-based agent to:
sign the first credential information with the second private key of the first service provider, and wherein it is the first credential information signed with the second private key of the first service provider that is encrypted with the first public key of the wallet client.

5. The method of claim 1, comprising:
receiving an identifier of one or more of the user or the terminal device of the user;
providing programs configured to deploy the wallet client to the terminal device of the user based on the received identifier; and
causing the wallet client to be deployed at the terminal device of the user, the wallet client including the digital identifier of the wallet client, the first public key and a first private key configured to decrypt a data encrypted using the first public key.

6. The method of claim 5, wherein the providing the programs configured to deploy the wallet client to the terminal device of the user includes sending a message to the terminal device, the message including a link configured to deploy the wallet client based on a selection by the user.

7. The method of claim 5, wherein the receiving the identifier of the one or more of the user or the terminal device of the user includes receiving the identifier through one or more of a portal application of the first client by human input or a service application of the first client from a first application of the first service provider that is different from the first client.

8. The method of claim 1, wherein the information of the user obtained from the first electronic health record system is vaccination information of the user.

9. The method of claim 1, wherein the wallet client is configured to identify the first client ad corresponding to the first service provider among a list of service providers.

10. The method of claim 1, wherein the information of the user includes personal identification information of the user.

11. The method of claim 1, comprising:
by the wallet client:
receiving, from a second service provider, a request for the first credential information;
sending a second digital identifier and a second public key of the wallet client to a second client of the second service provider;
identifying, by the wallet client, a second cloud-based agent as coupled to the second client of the second service provider;
encrypting signed first credential information with a second private key of the wallet client to generate signed and re-encrypted first credential information, the second private key of the wallet client corresponding to the second public key of the wallet client; and
sending, through the second cloud-based agent, the signed and re-encrypted first credential information to the second client.

12. The method of claim 11, wherein the second cloud-based agent decrypts the signed and re-encrypted first credential information using the second public key of the wallet client to obtain the signed first credential information and sends the signed first credential information to the second client.

13. The method of claim 12, further comprising:
by the second client:
obtaining, through the second cloud-based agent, a third public key of the first client from a distributed ledger; and
verifying authenticity of the signed first credential information using the third public key.

14. The method of claim 13, wherein the verifying authenticity of the signed first credential information is conducted by the second cloud-based agent.

15. A method, comprising:
by a first client deployed on a terminal device of a first service provider:
receiving, from a wallet client, a digital identifier of the wallet client;
sending the digital identifier of the wallet client to a first cloud-based agent and causing the first client to establish a secured connection with the wallet client, the establishing the secured connection with the wallet client including identifying a first public key stored at the first cloud-based agent as corresponding to a first private key of the wallet client; and
receiving, through the first cloud-based agent and from the wallet client, signed first credential information of a user that is issued by a second service provider,
wherein the first cloud-based agent:
receives from the wallet client signed and encrypted first credential information of the user,
decrypts the signed and encrypted first credential information of the user using the first public key to obtain signed first credential information,
obtains a second public key of the second service provider from a distributed ledger,
verifies authenticity of the signed first credential information using the second public key; and
sends the signed first credential information to the first client after the authenticity verification.

16. The method of claim 15, wherein the first client is coupled to a first electronic health record system, and the method comprises communicating, by the first client, the signed first credential information of the user to the first electronic health record system.

17. The method of claim 15, comprising:
receiving an identifier of one or more of the user or the terminal device of the user;
providing programs configured to deploy the wallet client to the terminal device of the user based on the received identifier; and
causing the wallet client be deployed at the terminal device of the user, the wallet client including the digital identifier of the wallet client, the first public key and the first private key configured to decrypt data encrypted using the first public key.

18. The method of claim 15, comprising:
by the first client:
invoking the first cloud-based agent to register the first service provider and to enable the wallet client to create a first key pair of the first private key and the first public key dedicated for communication with the first service provider.

19. A storage medium having executable instructions stored thereon, which when executed by a processor, configure the processor to implement acts comprising:
at a first client deployed on a terminal device of a first service provider:
receiving a request for secure connection from a wallet client deployed on a terminal device of a user, the request for secure connection including a first digital identifier of the wallet client, a first public key of the wallet client, and first personal identification information of the user;
in response to the request for secure connection:
verifying that the first personal identification information of the user is authentic; and
in response to verifying that the first personal identification information of the user is authentic, sending the digital identifier of the wallet client and the first public key of the wallet client to a first cloud-based agent to enable the first cloud-based agent to store the digital identifier of the wallet client together with the first public key of the wallet client;
receiving a request to issue a credential that identifies a credential template; and
in response to receiving the request to issue a credential:
accessing the identified credential template;
retrieving information of the user from a first electronic health record system coupled to the first client;
populating the accessed credential template using the information of the user retrieved from the first electronic health record system to generate first credential information; and
sending the first credential information with the wallet client's digital identifier to the first cloud-based agent to enable the first cloud-based agent to:
encrypt the first credential information with the first public key of the wallet client; and
forward the encrypted first credential information to the wallet client using the wallet client's digital identifier.

20. The storage medium of claim 19, wherein the acts further comprise:
at the first client:
invoking the first cloud-based agent to register the first service provider, to enable the first cloud-based agent to:
create a second key pair for the first service provider comprising a second private key and second public key;
store the second private key; and
cause publication of the second public key in a distributed ledger, and wherein the sending the first credential information with the wallet client's digital identifier further enables the first cloud-based agent to:
sign the first credential information with the second private key of the first service provider, and wherein it is the first credential information signed with the second private key of the first service provider that is encrypted with the first public key of the wallet client.
